(19) United States
(12) Patent Application Publication
(10) Pub. No.: US 2004/0266704 A1
(43) Pub. Date: Dec. 30, 2004

(54) Method comprising irinotecan for treatment of breast cancer

(76) Inventors: Langdon L. Miller, Lebanon, NJ (US); David Emanuel, Tenafly, NJ (US); James Patrick McGovren, Kalamazoo, MI (US); Larry J. Schaaf, Dublin, OH (US); Sumant Ramachandra, East Brunswick, NJ (US)

Correspondence Address: Patrick G. Gattari, McDonnell Boehnen Hulbert & Berghoff LLP, 32nd Floor, 300 S. Wacker Drive, Chicago, IL, 60606, US

(21) Appl. No.: 10/832,794

(22) Filed: Apr. 26, 2004

Related U.S. Application Data
(60) Provisional application No. 60/466,222, filed on Apr. 28, 2003.

(51) Int. Cl. A61K 31/7072; A61K 31/704; A61K 31/4745

(52) U.S. Cl. 514/34; 514/283; 514/50

(57) ABSTRACT

The present invention relates to a method for treating locally advanced or metastatic breast cancer in a patient who demonstrated failure of prior treatment with an anthracycline, a taxane and a fluoropyrimidine, which comprises administering a therapeutically effective amount of irinotecan.

### FIELD OF THE INVENTION

The present invention relates to the use of irinotecan for the treatment of breast cancer, in particular to a method for treating patients with breast cancer after failure of prior anthracycline, taxane and fluoropyrimidine-containing chemotherapy.

### BACKGROUND OF THE INVENTION

Approximately 16,400 women, at any point in time, in the United States are living with relapsed or refractory metastatic breast cancer after failure of all three core chemotherapy agents generally utilized for the treatment of breast cancer, i.e. an anthracycline (such as, e.g. doxorubicin or epirubicin), a taxane (such as, e.g. paclitaxel or docetaxel) and a fluoropyrimidine (such as, e.g. 5-fluorouracil or capecitabine). It is customary clinical practice for patients with metastatic breast cancer to receive multiple chemotherapeutic agents in sequence in order to maintain control of tumor growth for as long as possible. The only drug registered as “3^{rd}-line” therapy of metastatic breast cancer, after failure of a taxane and anthracycline, is capecitabine, which is approved for use in patients who demonstrated resistance to both a taxane and an anthracycline-containing regimen or to paclitaxel alone and for whom further use of an anthracycline is contraindicated. Resistance is defined as disease progression on treatment, with or without an initial response or relapse within 6 months of completing treatment with an anthracycline-containing adjuvant regimen. When patients relapse after receiving capecitabine monotherapy or in combination with docetaxel after they have failed an anthracycline-based regimen they have limited options. Patients requesting or requiring treatment in this setting are generally offered “off-label” monotherapy or drug combinations as palliative therapy. None of these agents have been evaluated in a controlled study in this patient population. Additionally, physicians prescribe them using a variety of dosages and schedules, generally reflecting local or regional preference and prescribing practice.

Thus, the subpopulation of patients with locally advanced or metastatic breast cancer that has failed an anthracycline, a taxane and a fluoropyrimidine and who still require further treatment represents a unique cohort for which no currently available drug has been registered for use in the United States. There is therefore an unmet medical need for new agents that are specifically targeted to this unique subset of patients.

Schoemaker N. et al., reported the results of a phase I trial with an oral formulation (powder-filled capsule) of CPT-11. This study was conducted in 34 patients with colorectal cancer (28), other gastro-intestinal cancers (4), non-small cell lung cancer (NSCLC) (1) and ovarian cancer (1) (Abstract 295, Proc. ASCO 2001).

Dumez H. et al., reported the results of a phase I trial with an oral formulation (powder-filled capsule) of CPT-11. This study was conducted in 46 patients with melanoma (10), colorectal cancer (5), genito-urinary tract cancer (6), lung cancer (4), thyroid cancer (3), liver cancer (3), pancreatic cancer (2), breast cancer (2) and other cancer types (11) (Abstract 408, Proc. ASCO 2001).

Pitot H. C. et al., reported the results of a phase I trial with an oral formulation (powder-filled capsule) of CPT-11 given daily for 5 days every 3 weeks in patients with advanced solid tumors (Abstract 401, Proc. ASCO 2001).

Sharma S. et al., reported the results of a phase I trial with an oral formulation (powder-filled capsule) of CPT-11 given daily for 14 days every 3 weeks to patients with advanced solid tumors (Abstract 407, Proc. ASCO 2001). The trial evaluated MTD, DLTs, safety profile, and PK of a powder filled capsule formulation of oral CPT-11.

Drengler R. et al., described a phase-I trial of with intravenous solution CPT-11 administered orally in CranGrape juice. The trial evaluated the maximum-tolerated dose (MTD), dose-limiting toxicities (DLTs), pharmacokinetic profile, and antitumor effects in 28 patients with colorectal cancer, endometrial cancer and renal cancer (Journal of Clinical Oncology, Vol. 17, No. 2, 199: pp 685-696).

Taguchi T. et al., reported the results of an early phase II study of intravenous CPT-11 in patients with advanced breast cancer. The results of this study suggested that CPT-11 administered by intravenous drip-infusion was effective against advanced or recurrent breast cancer (Gan to Kagaku Ryoho, Japanese Journal of Cancer & Chemotherapy, 21(1):83-90, 1994 January, English abstract). Results of a second phase II study, in which Taguchi T. et al. evaluated the efficacy of intravenous CPT-11 in patients who received prior chemotherapy for advanced breast cancer, confirmed that CPT-11 was a promising drug in patients with prior endocrine therapy and prior chemotherapy including adriamycin or other anthracyclines (Gan to Kagaku Ryoho, Japanese Journal of Cancer & Chemotherapy, 21(7):1017-24, 1994 June, English abstract).

Doihara H. et al., described four cases of recurrent breast cancer effectively treated by intravenous CPT-11. These cases had undergone previous chemotherapy including doxorubicin. The results suggested that CPT-11 was an effective agent against advanced or recurrent breast cancer, and especially useful for patients who had developed a tolerance to previous therapies (Gan to Kagaku Ryoho, Japanese Journal of Cancer & Chemotherapy, 21(8):1263-6, 1994 July, English abstract).

Ikeda H. et al., reported the results of a pilot study of intravenous CPT-11, as a salvage therapy, for metastatic breast cancer. In this study, twelve metastatic breast cancer patients were treated with CPT-11. All patients had received prior chemotherapy including an anthracycline. In spite of intense prior chemotherapy, the treatment results with CPT-11 were satisfactory for anthracycline resistant metastatic breast cancer (Gan to Kagaku Ryoho, Japanese Journal of Cancer & Chemotherapy 27(5):723-7, 2000 May, English abstract).

In a review article, Shigeoka Y. et al., expressed a negative opinion on the clinical efficacy of intravenous CPT-11 in advanced and metastatic breast cancer patients previously treated with doxorubicin- and docetaxel-containing regimens. Even if previous phase II trials in Japan suggested that CPT-11 was a promising agent for advanced or metastatic breast cancer pretreated with anthracycline, they noticed that CPT-11 evaluated in the salvage setting was inactive against advanced and metastatic breast cancer pretreated with doxorubicin and docetaxel (Japanese Journal of Clinical Oncology, 31(8):370-4, 2001 August, abstract).

The North Central Cancer Treatment Group (NCCTG) reported preliminary results of a randomized phase II study comparing two schedules of IV CPT-11 (NCCTG 96-32-55) in 103 patients with refractory advanced breast cancer who had failed prior chemotherapy with an anthracycline, a taxane or both drugs. The authors concluded that these preliminary data indicate that CPT-11 is an active agent in this advanced patient population with an acceptable toxicity profile (Abstract 206, Proc. ASCO 2002).

It would be apparent from the foregoing that CPT-11 has achieved inconsistent results in breast cancer patients pre-treated with anthracyclines and taxanes. In addition, nothing has been reported or suggested as to the efficacy of CPT-11 once a patient failed not only an anthracycline and a taxane, but also failed prior therapy with a fluoropyrimidine.

It has now been found, and this forms the subject of the present invention, that patients with advanced breast cancer who have failed prior therapy with an anthracycline, a taxane and a fluoropyrimidine and who still desire or require further treatment, could realize significant clinical benefit by having access to irinotecan.

### DESCRIPTION OF THE INVENTION

It is therefore a first object of the present invention a method for treating locally advanced or metastatic breast cancer in a patient who demonstrated failure of prior treatment with an anthracycline, a taxane and a fluoropyrimidine, which comprises administering a therapeutically effective amount of irinotecan.

Further, the present invention relates to the use of irinotecan for the preparation of a medicament for treating locally advanced or metastatic breast cancer in patients who demonstrated failure of prior treatment with an anthracycline, a taxane and a fluoropyrimidine.

Irinotecan [1,4′-Bipiperidine]-1′-carboxylic acid (4S)-4,11-diethyl-3,4,12,14-tetrahydro-4-hydroxy-3,14-dioxo-1H-pyrano[3′,4′:6,7]indolizino[1,2-b]quinolin-9-yl ester is a camptothecin analog and topoisomerase-I inhibitor derived from a compound, which occurs naturally in the Chinese tree, Camptotheca acuminata. Irinotecan can be prepared following the procedure disclosed in U.S. Pat. No. 4,604,463, European patent No. 835,257 or S. Sawada et al., Chem. Pharm. Bull. 39,1446 (1991). Irinotecan hydrochloride, clinically investigated as CPT-11, is a commercially available compound (CAMPTOSAR™, Pharmacia Corp.).

As used herein, the term “irinotecan” encompasses all pharmaceutically acceptable salts of irinotecan, particularly the hydrochloride salt.

In the present specification “anthracycline” means, unless otherwise specified, doxorubicin or epirubicin.

In the present specification “taxane” means, unless otherwise specified, paclitaxel or docetaxel.

In the present specification “fluoropyrimidine” means, unless otherwise specified, 5-fluorouracil (5-FU) or capecitabine.

Preferably, irinotecan may be administered orally in the form of a pharmaceutically acceptable formulation for oral administration, which can provide a means for protracted drug exposure to actively cycling malignant cells with greater convenience and benefit to patients. In general, the pharmaceutically acceptable formulations for oral administration according to the present invention may comprise a therapeutically effective amount of irinotecan in combination with a pharmaceutically acceptable carrier or diluent. Examples of oral formulations include solid oral preparations such as, e.g., tablets, capsules, powders and granules, and liquid oral preparations such as e.g., solutions and suspensions, that may be prepared following conventional literature or common techniques well known to those skilled in the art.

Suitable oral dosage forms according to the present invention may be prepared, for example, as described in the Pharmacia & Upjohn S.p.A. International patent application WO 01/10443 filed on Jul. 11, 2000, Teva Pharm. Ind. LTD U.S. patent application Ser. No. 20020147208 filed on Dec. 20, 2001 and Pharmacia Italia S.p.A. International patent application WO 01/30351 filed on Oct. 2, 2000.

The dosage regimen should be preferably tailored to the patient's conditions and response in a manner that is conventional for any therapy, and may need to be adjusted in response to changes in conditions.

For example, an oral formulation of irinotecan may be administered, according to the invention, daily for 5 days every 3 weeks to adult patients at doses ranging from 30 to 90 mg/m² (based on body surface area) or daily for 14 days every 3 weeks at doses ranging from 15 to 45 mg/m² (based on body surface area). Preferably, an oral formulation of irinotecan may be administered, according to the invention, daily for 5 days every 3 weeks to adult patients at doses ranging from 50 to 70 mg/m2 (based on body surface area) or daily for 14 days every 3 weeks at doses ranging from 25 to 35 mg/m² (based on body surface area). More preferably, an oral formulation of irinotecan may be administered, according to the invention, daily for 5 days every 3 weeks to adult patients at a dose of 60 mg/m² or 70 mg/m² (based on body surface area) or daily for 14 days every 3 weeks at a dose of 30 mg/m² (based on body surface area).

In the present specification the term “failure of treatment” includes progression of disease while receiving a chemotherapy regimen without experiencing any transient improvement, no objective response after receiving one or more cycles of a chemotherapy regimen, a limited response with subsequent progression, while receiving a chemotherapy regimen or significant toxicity following treatment or attainment of the maximum cumulative dose that would preclude further treatment.

In the present specification “therapeutically effective amount” means, unless otherwise indicated, the amount of drug that is required to be administered to achieve the desired therapeutic effect.

In the present specification “adjuvant therapy” means, unless otherwise specified, a treatment given after the primary treatment to increase the chances of a cure. Adjuvant therapy may include chemotherapy, radiation therapy, hormone therapy, or biological therapy.

In the present specification “response rate” means, unless otherwise specified, the percentage of patients whose cancer shrinks or disappears after treatment.

In the present specification “complete response” means, unless otherwise specified, the disappearance of all signs of cancer in response to treatment. This does not always mean the cancer has been cured.

In the present specification “partial response” means, unless otherwise specified, a decrease in the size of a tumor, or in the extent of cancer in the body, in response to treatment.

In the present specification “refractory cancer” means, unless otherwise specified, a cancer that has not responded to treatment.

In the present specification “regimen” means, unless otherwise specified, a treatment plan that specifies the dosage, the schedule, and the duration of treatment.

In the present specification “relapse” means, unless otherwise specified, the return of signs and symptoms of cancer after a period of improvement.

In the present specification “palliative therapy” means, unless otherwise specified, a treatment given to relieve symptoms caused by advanced cancer. Palliative therapy does not alter the course of a disease but can improve the quality of life.

The efficacy and safety of oral CPT-11 according to the present invention can be illustrated by the example below.

### EXAMPLE

The efficacy and safety of two different schedules of oral CPT-11 is evaluated in patients with metastatic breast cancer after failure of prior anthracycline, taxane and fluoropyrimidine-containing chemotherapy (ATF failure). The primary objective of the study is determination of the confirmed objective tumor response rate of two different schedules of administration of CPT-11. The secondary objectives include evaluation of tumor control and overall survival, determination of the overall safety profile of each schedule of treatment regimen.

Patients receive CPT-11 in one of the following treatment regimens:

| Drug | Starting dose | Dosing interval | Planned duration |
|---|---|---|---|
| Oral CPT-11 | 60 mg/m²/day PO | Days 1-5 every | Until disease |
| | | 3 weeks | progression or |
| Oral CPT-11 | 30 mg/m²/day PO | Days 1-14 every | unacceptable |
| | | 3 weeks | toxicity |

1. A method for treating locally advanced or metastatic breast cancer in a patient who demonstrated failure of prior treatment with an anthracycline, a taxane and a fluoropyrimidine, which comprises a administering a therapeutically effective amount of irinotecan.

2. A method according to claim 1, wherein irinotecan is in the form of its hydrochloride salt.

3. A method according to claim 1, wherein an anthracycline is doxorubicin or epirubicin.

4. A method according to claim 1, wherein a taxane is paclitaxel or docetaxel.

5. A method according to claim 1, wherein a fluoropyrimidine is 5-fluorouracil or capecitabine.

6. A method according to claim 1, wherein irinotecan is to be orally administered.

7. A method according to claim 6, wherein irinotecan is to be administered daily for 5 days every 3 weeks to adult patients at doses ranging from 30 to 90 mg/m² (based on body surface area).

8. A method according to claim 6, wherein irinotecan is to be administered daily for 14 days every 3 weeks at doses ranging from 15 to 45 mg/m² (based on body surface area).

9. A method according to claim 7, wherein irinotecan is to be administered daily for 5 days every 3 weeks at doses ranging from 50 to 70 mg/m² (based on body surface area).

10. A method according to claim 8, wherein irinotecan is to be administered daily for 14 days every 3 weeks at doses ranging from 25 to 35 mg/m² (based on body surface area).

11. A method according to claim 9, wherein irinotecan is to be administered at a dose of 60 mg/m² (based on body surface area).

12. A method according to claim 9, wherein irinotecan is to be administered at a dose of 70 mg/m² (based on body surface area).

13. A method according to claim 10, wherein irinotecan is to be administered at a dose of 30 mg/m² (based on body surface area).

14. Use of irinotecan for the preparation of a medicament for treating locally advanced or metastatic breast cancer in patients who demonstrated failure of prior treatment with an anthracycline, a taxane and a fluoropyrimidine.

15. Use according to claim 14, wherein irinotecan is in the form of its hydrochloride salt.

16. Use according to claim 14, wherein an anthracycline is doxorubicin or epirubicin.

17. Use according to claim 14, wherein a taxane is paclitaxel or docetaxel.

18. Use according to claim 14, wherein a fluoropyrimidine is 5-fluorouracil or capecitabine.

19. Use according to claim 14, wherein irinotecan is to be orally administered.

20. Use according to claim 19, wherein irinotecan is to be administered daily for 5 days every 3 weeks to adult patients at doses ranging from 30 to 90 mg/m² (based on body surface area).

21. Use according to claim 19, wherein irinotecan is to be administered daily for 14 days every 3 weeks at doses ranging from 15 to 45 mg/m² (based on body surface area).

22. Use according to claim 20, wherein irinotecan is to be administered daily for 5 days every 3 weeks at doses ranging from 50 to 70 mg/m² (based on body surface area).

23. Use according to claim 21, wherein irinotecan is to be administered daily for 14 days every 3 weeks at doses ranging from 25 to 35 mg/m² (based on body surface area).

24. Use according to claim 22, wherein irinotecan is to be administered at a dose of 60 mg/m² (based on body surface area).

25. Use according to claim 22, wherein irinotecan is to be administered at a dose of 70 mg/m² (based on body surface area).

26. Use according to claim 23, wherein irinotecan is to be administered at a dose of 30 mg/m² (based on body surface area).
